(19) United States
(12) Patent Application Publication
(10) Pub. No.: US 2016/0250108 A1
(43) Pub. Date: Sep. 1, 2016

(54) VISCOSIFIER COMPRISING FILAMENTOUS POLYMER PARTICLES

(71) Applicants: ARKEMA FRANCE, Colombes (FR); UNIVERSITE CLAUDE BERNARD LYON 1, Villeurbanne (FR); CENTRE NATIONAL DE LA RECHERCHE SCIENTIFIQUE, Paris (FR)

(72) Inventors: Bernadette CHARLEUX, Lyon (FR); Franck D'AGOSTO, Villeurbanne (FR); Raber INOUBLI, Pau (FR); Stephanie MAGNET, Morlanne (FR)

(73) Assignees: ARKEMA FRANCE, Colombes (FR); UNIVERSITE CLAUDE BERNARD LYON 1, Villeurbanne (FR); CENTRE NATIONAL DE LA RECHERCHE SCIENTIFIQUE, Paris (FR)

(21) Appl. No.: 14/906,731

(22) PCT Filed: Jul. 23, 2014
(86) PCT No.: PCT/FR2014/051906

§ 371 Date: Jan. 21, 2016

(30) Foreign Application Priority Data
Jul. 25, 2013 (FR) 1357360

(51) Int. Cl. A61K 8/02; A61Q 19/00; C09D 133/14; C09D 7/00; C09D 7/12; A61K 8/81; A61K 8/90

(52) U.S. Cl. CPC A61K 8/027; A61K 8/8152; A61Q 19/00; A61K 8/90; C09D 7/002; C09D 7/1291; C09D 7/1275; C09D 133/14; C09D 7/125; A61K 2800/48; A61K 2800/10

(57) ABSTRACT

The invention relates to the use of polymer particles in the form of filaments formed by block copolymers, as viscosifiers or agents that modify the rheology of organic or aqueous solutions. More specifically, the invention relates to the use of said crosslinked filamentous polymer particles for improving the resistance to heat ageing of an organic or aqueous solution.

The present invention relates to the use of polymer particles in the form of filaments consisting of block copolymers as viscosifiers or rheology modifiers for aqueous or organic solutions. More particularly, the invention relates to the use of said crosslinked filamentous polymer particles for improving the resistance to thermal aging of an aqueous or organic solution.

It is known by a person skilled in the art that an increase in the viscosity of an aqueous solution with very small contents of additive can be obtained by the use of water-soluble polymers of very high molar mass and/or having charged monomer units (in particular monomer units charged by acid groups) or by the use of hydrophilic biopolymers giving rigid structures.

Charged water-soluble polymers (such as high molecular weight polyacrylamides (HPMAs), which are acrylamides copolymerized with an ionic monomer) exhibit a viscosifying nature via the large increase in the radius of gyration of the molecule brought about by the repulsive interactions of the charges present in the molecule. The presence of salts or a variation in pH of the medium can “screen” these charges, suppress these interactions and thus suppress the viscosifying effect. Furthermore, these polymers have a tendency to decompose at temperatures greater than 90° C. and thus to lose their rheological properties.

Hydrophilic biopolymers, such as scleroglucan, are very effective rheology modifiers but exhibit a very high sensitivity to bacterial degradation. These molecules are degraded by certain microorganisms and thus lose all viscosifying and shear-thinning properties.

Other polymer compounds have been used as rheology modifiers, for example hydrophobically associative polymers (HAPs), which have a hydrophilic backbone and comprise, along the chains, small amounts of hydrophobic monomers capable of joining together in water in the form of hydrophobic nanodomains. These act as temporary crosslinking points and confer a marked shear-thinning nature on the HAPs.

Novel polymer compounds capable of modifying the rheology of aqueous or organic solutions and which overcome the disadvantages presented above have been provided by the applicant company in the applications WO 2012/085415 and WO 2012/085473. These documents describe filamentous polymer structures capable of retaining their morphology after significant dilution in water and/or in an organic solvent. These polymer particles in the form of filaments consisting of block copolymers exhibit a viscosifying and shear-thinning nature in a dispersed medium, this being the case at a very low concentration. Furthermore, the viscosifying and shear-thinningeffect of said filamentous particles is not affected by the presence of salt or by the variations in pH of the medium and said particles are not sensitive to bacterial degradation.

It has now been found that a composition comprising said filamentous polymer particles which are obtained in the presence of a crosslinking agent exhibits an increased resistance to thermal aging, which makes it possible for it to retain its rheological behavior for a longer time and within a broader temperature range than the compositions of the prior art, this being the case even at a very low content of said particles.

A subject matter of the invention is the use of a latex of filamentous polymer particles consisting of block copolymers synthesized by controlled radical emulsion polymerization for improving the resistance to thermal aging of an aqueous or organic solution. Characteristically, these polymer particles are crosslinked and are provided in the form of cylinders having a length/diameter ratio of greater than 100. A composition obtained by the addition of at least 100 ppm, preferably from 500 to 10 000 ppm, of these particles to an aqueous or organic solution will retain its viscosifying nature for several days at a temperature of greater than 100° C., whereas compositions based on noncrosslinked filamentous polymer particles lose their viscosifying nature after one day at more than 100° C. due to the degradation of said noncrosslinked particles.

According to one embodiment, the use of said latex of crosslinked filamentous polymer particles at a content by weight of 1% makes it possible to retain the viscosifying nature of a composition for 4 days at 140° C.

According to one embodiment, the synthesis of said particles is carried out starting from at least one hydrophobic monomer and a crosslinking agent in the presence of a living macroinitiator derived from a nitroxide, characterized in that:
said filamentous particles are obtained in an aqueous medium directly during the synthesis of said block copolymers carried out by heating the reaction medium at a temperature of 60 to 120° C.,
said macroinitiator is water-soluble,
the percentage of the molar mass of the water-soluble macroinitiator in the final block copolymer is between 10 and 50%, and of that:
the degree of conversion of the hydrophobic monomer is at least 50%.

This direct method for the preparation of crosslinked filamentous particles does not require the use of an organic cosolvent.

In the context of the present invention, the term “filamentous particles” corresponds to assemblies of amphiphilic macromolecules which, when they are in suspension in water (in other words, when they form an aqueous dispersion), take the form of filaments (in other words, of solid and flexible cylinders), the core of which consists of the hydrophobic components and the surface of which consists of the hydrophilic components of said macromolecules. These filamentous particles can be observed with a transmission electron microscope (TEM). The microscopy images show filaments, the diameter of which is greater than or equal to 5 nm and the length of which is greater than 500 nm, preferably greater than 1 micron, advantageously greater than 5 microns. According to one embodiment, the length of the filamentous particles according to the invention is at least 10 micrometers.

According to another embodiment, the synthesis of said filamentous particles is carried out by radical polymerization by reversible addition/fragmentation chain transfer (RAFT) in water in the presence of a hydrophilic macromolecular RAFT agent (or RAFT macroagent).

The compositions targeted by the present invention are obtained by the addition of said crosslinked filamentous polymer particles to an aqueous or organic solution at a content by weight of a minimum of 100 ppm, preferably of 500 to 10 000 ppm. Said compositions, the resistance of which to aging, in particular to thermal aging, is improved, are particularly suitable for the reinforced extraction of hydrocarbons. To this end, the composition according to the invention containing at least 500 ppm of said particles and mixed with water or with brine is injected under pressure into the rock. Other applications of these compositions are targeted at the cosmetics field, the paints field and thickeners.

The invention and the advantages which it provides will be better understood in the light of the detailed description which will follow and of the appended FIG. 1, which illustrates the effect of the thermal aging on the rheological behavior of an aqueous composition comprising crosslinked filamentous polymer particles, in comparison with an aqueous composition comprising noncrosslinked filamentous polymer particles.

The subject matter of the present invention relates to the rheological properties (viscosifying and shear-thinningnature) in a dispersed medium of copolymer particles having a very specific elongated fibril shape. It has now been found that a composition comprising said crosslinked filamentous polymer particles, even at very low concentrations, exhibits an increased resistance to aging, in particular to thermal aging, which makes it possible for it to retain its rheological behavior for a longer time and within a broader temperature range than the compositions of the prior art.

The viscosifying nature at very low concentrations is contributed by a pseudo-percolation of the structure, obtained at very low concentrations, in the dispersed medium. The shear-thinningnature is obtained by a pseudo-disentangling obtained very rapidly (as a function of the strain or shear gradient) by virtue of the stiffness and of the very high ratio of the length to the radius of the structure. Furthermore, by virtue of its gelled nature, this copolymer structure is not sensitive to the salinity or to variations of pH of the aqueous or organic medium to be viscosified.

The term “shear thinning” is understood to mean the decrease in the rheological properties (viscosity) under the effect of an increase in the stress, in the shearing or in the strain applied to the system studied.

To this end, a subject matter of the invention is the use of a latex of filamentous polymer particles consisting of block copolymers synthesized by controlled radical emulsion polymerization for improving the resistance to thermal aging of an aqueous or organic solution. Characteristically, these polymer particles are crosslinked and are provided in the form of cylinders having a length/diameter ratio greater than 100. The term “latex” is understood here to mean a continuous aqueous or organic phase in which are dispersed the filamentous polymer fibers or particles consisting of block copolymers.

According to one embodiment, the synthesis of said particles is carried out starting from at least one hydrophobic monomer and a crosslinking agent in the presence of a living macroinitiator derived from a nitroxide.

Characteristically, said crosslinked filamentous particles are obtained in an aqueous medium for the synthesis of said block copolymers carried out by heating the reaction medium at a temperature of 60 to 120° C., with a percentage of the molar mass of the hydrophilic macroinitiator in the final block copolymer of between 10 and 50%, the degree of conversion of the hydrophobic monomer and crosslinking agent being at least 50%. The crosslinking agent is advantageously introduced into the reaction medium at a content of at least 1% by weight and preferably between 5 and 15% by weight, with respect to the weight of hydrophobic monomer. The initial pH of the aqueous medium can vary between 5 and 10. This direct method for the preparation of crosslinked filamentous particles does not require the use of an organic cosolvent.

The term “living macroinitiator” is understood to mean a polymer comprising at least one end capable of being reengaged in a polymerization reaction by addition of monomers at an appropriate temperature and an appropriate pressure. Advantageously, said macroinitiator is prepared by CRP. The term “water-soluble macroinitiator” is understood to mean a water-soluble polymer comprising, at its end, a reactive functional group capable of reinitiating a radical polymerization. This macroinitiator is predominantly composed of hydrophilic monomers, that is to say monomers exhibiting one or more functional groups capable of establishing hydrogen bonds with water. In the case of the polymerization of a hydrophobic monomer, an amphiphilic copolymer will be formed, the hydrophilic block of which will consist of the macroinitiator while the hydrophobic block of which will result from the polymerization of the hydrophobic monomer(s). According to an alternative embodiment, said preformed water-soluble macroinitiator is added to the reaction medium comprising at least one hydrophobic monomer.

According to another alternative form within the first embodiment, said water-soluble macroinitiator is synthesized in the aqueous reaction medium in a preliminary stage, without isolation of the macroinitiator formed or removal of the possible residual hydrophilic monomers. This second alternative form is a one-pot polymerization.

The hydrophobic monomers can be chosen from:
vinylaromatic monomers, such as styrene or substituted styrenes,
alkyl, cycloalkyl or aryl acrylates, such as methyl acrylate, ethyl acrylate, butyl acrylate, 2-ethylhexyl acrylate or phenyl acrylate,
alkyl, cycloalkyl, alkenyl or aryl methacrylates, such as methyl methacrylate, butyl methacrylate, lauryl methacrylate, cyclohexyl methacrylate, allyl methacrylate, 2-ethylhexyl methacrylate or phenyl methacrylate,
and vinylpyridine.

These hydrophobic monomers are added to the reaction medium, which predominantly comprises water.

The crosslinking agent employed is a crosslinking comonomer other than the abovementioned hydrophobic monomers.

The term “crosslinking comonomer” is understood to mean a monomer which, due to its reactivity with the other monomers present in the polymerization medium, is capable of generating a covalent three-dimensional network. From a chemical viewpoint, a crosslinking comonomer generally comprises at least two polymerizable ethylenic functional groups which, on reacting, are capable of creating bridges between several polymer chains.

These crosslinking comonomers may be capable of reacting with the unsaturated hydrophobic monomers during the synthesis of said particles.

Mention may be made, among the crosslinking comonomers, of divinylbenzenes, trivinylbenzenes, allyl (meth)acrylates, diallyl maleate polyol (meth)acrylates, such as trimethylolpropane tri(meth)acrylates, alkylene glycol di(meth)acrylates which have from 2 to 10 carbon atoms in the carbon-based chain, such as ethylene glycol di(meth)acrylates, 1,4-butanediol di(meth)acrylates or 1,6-hexanediol di(meth)acrylates, or N,N′-alkylenebisacrylamides, such as N,N′-methylene-bisacrylamide. Preferably, use will be made, as crosslinking agent, of divinylbenzene or a dimethacrylate.

The use of said process makes it possible to obtain crosslinked filamentous polymer particles in which the content by weight of the hydrophilic part making up the block copolymer is less than 25%.

Characteristically, the crosslinked filamentous particles according to the invention exhibit a percentage of the molar mass of the hydrophilic macroinitiator in the final block copolymer of between 10 and 50%. Preferably, the percentage of the molar mass of the water-soluble macroinitiator in the final block copolymer is between 10 and 30%.

As observed by TEM, the crosslinked filamentous particles according to the invention are provided in the form of cylindrical fibers having a length/diameter ratio of greater than 100; their diameter is unvarying over the whole of their length and is greater than or equal to 5 nm, while their length is greater than 500 nm, preferably greater than 1 micron, advantageously greater than 5 microns and more preferably still greater than or equal to 10 micrometers. The filamentous particles according to the invention experience a maintenance in their shape and their structure in a dispersed medium, independently of their concentration in the medium and/or of the variations in pH or in salinity of the latter.

According to a second embodiment, the synthesis of said crosslinked filamentous particles is carried out by radical polymerization by reversible addition/fragmentation chain transfer (RAFT) in water in the presence of a hydrophilic macromolecular RAFT agent (or RAFT macroagent).

The invention will now be described with the help of the following examples, given by way of illustration and without limitation.

### EXAMPLE 1

### Preparation of the Crosslinked Filamentous Polymer Particles According to the Invention (Sample A)

This example illustrates the preparation of a living poly(methacrylic acid-co-sodium styrenesulfonate) copolymer, used as macroinitiator, control agent and stabilizing agent, as vessel, heel for the synthesis of hairy nanoparticles in the form of crosslinked filamentous micelles of poly(sodium methacrylate-co-sodium styrenesulfonate)-b-poly(n-butyl methacrylate-co-styrene) block copolymers.

The amphiphilic copolymer is synthesized in a single stage.

The conditions for the synthesis of the macroinitiator can be varied (duration of the polymerization, content of sodium styrenesulfonate, concentration and pH) in order to adjust and vary the composition of the macroinitiator.

In order to do this, a mixture containing 6.569 g of methacrylic acid (0.84 mol.l_{aq}⁻¹ or 0.79 mol.l⁻¹), 1.444 g of sodium styrenesulfonate (6.97×10⁻² mol.l_{aq}⁻¹ or 6.51×10⁻² moll ⁱ, i.e. f_{0,SS}=0.076; f_{0,SS}=n_{SS}/(n_{SS}+N_{MAA})), 0.3594 g of Na₂CO₃ (3.75×10⁻² mol.l_{aq}⁻¹ or 3.50×10⁻² mol.l⁻¹) and 87.1 g of deionized water is degassed at ambient temperature by bubbling with nitrogen for 15 min. At the same time, 0.3162 g (9.18×10⁻³ mol.l_{aq}⁻¹ or 8.57×mol.l_{aq}⁻¹) of the alkoxyamine BlocBuilder®-MA is dissolved in 3.3442 g of 0.4 M sodium hydroxide solution (1.6 equivalent, with respect to the methacrylic acid units of the BlocBuilder®-MA) and degassed for 5 minutes.

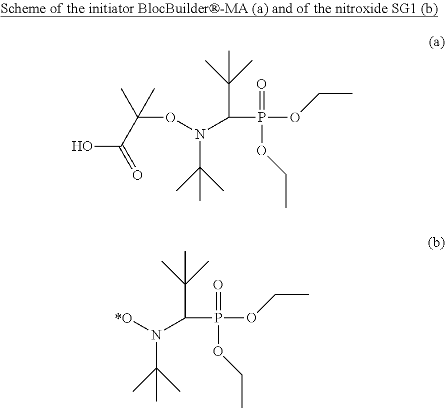

The BlocBuilder®-MA solution is introduced into the reactor at ambient temperature with stirring at 250 rpm. The monomer solution is subsequently introduced slowly into the reactor. The reactor is subjected to a nitrogen pressure of 1.1 bar, still with stirring. The time t=0 is launched when the temperature reaches 60° C. The temperature of the reaction medium reaches 65° C. after 15 minutes.

During this reaction, 18.01 g of n-butyl methacrylate and 2.01 g of styrene are introduced into an Erlenmeyer flask (solids content=24%) and the mixture is degassed by bubbling with nitrogen at ambient temperature for 10 minutes.

After 15 minutes of synthesis, that is to say the synthesis of the macroinitiator of the poly(methacrylic acid-co-sodium styrenesulfonate)-SG1 type, the second reaction medium, containing the hydrophobic monomers, is introduced at ambient pressure, then a nitrogen pressure of 3 bar and stirring at 250 rpm. The reactor is maintained at 90° C. throughout the polymerization.

After 54 minutes, 2.06 g of ethylene glycol dimethacrylate (f_{0,EGDMA}=0.066 mol) (f_{0,EGDMA}=n_{EGDMA}/(n_{EGDMA}+n_{BuMA}+n_{Sry}) (solids content=25%) are introduced into the reactor in order to crosslink the fibers after they are formed.

Samples are taken at regular intervals in order to determine the kinetics of polymerization by gravimetry (measurement of solids content).

The characteristics of the latexes withdrawn from the second stage of the synthesis of the nanoparticles are presented in table 1 below.

TABLE 1

| Time | Conversion | |
|---|---|---|
| (h) | (%) | pH |
| 0.25 | 6.4 | — |
| 0.58 | 34.6 | 4.41 |
| 0.9 | 66.1 | — |
| 1.25 | 88.7 | — |
| 3.0 | 94.9 | 4.54 |

The diameter of the fibers, measured by Transmission Electron Microscopy TEM (ImageJ software), is 45.3 nm. This microscope is of JEOL 100 Cx II type at 100 keV equipped with a high resolution CCD camera, Keen View camera from SIS.

### EXAMPLE 2

### Comparative: Preparation of the Noncrosslinked Filamentous Polymer Particles (Sample B)

This example illustrates the synthesis of filamentous particles of poly(sodium methacrylate-co-sodium styrenesulfonate)-b-poly(methyl methacrylate-co-styrene) block copolymers from the poly(sodium methacrylate-co-sodium styrenesulfonate) macroinitiator prepared as follows:

A mixture containing 75.2 g of methacrylic acid (2.0 mol.l⁻¹), 17.32 g of sodium styrenesulfonate (0.18 mol.l⁻¹, i.e. f_{0,SS}=0.087) and 398 g of DMSO is degassed at ambient temperature by bubbling with nitrogen. 3.782 g (2.27×10⁻² mol.l⁻¹) of the alkoxyamine BlocBuilder®-MA (N-(2-methylpropyl)-N-(1-diethylpho sphono-2,2-dimethylpropyl)-O-(2-carboxylprop-2-yl)hydroxylamine) are subsequently added.

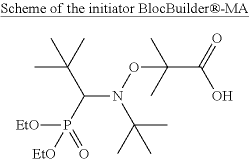 [SMILES: CCOP(=O)(OCC)C(N(OC(C)(C)C(=O)O)C(C)(C)C)C(C)(C)C]

The degassing is continued for 10 minutes. The degassed mixture is introduced into a 1 l three-necked flask, preheated to 75° C., surmounted by a reflux condenser equipped with a bubbler, a nitrogen inlet and a thermometer. The polymerization is carried out at 76° C. and the time t=0 is launched when the temperature reaches 35° C. in the reaction medium. The macroinitiator obtained is P(MAA-co-SS)-SG₁. The handling is halted after 16 minutes of reaction by immersing the medium with stirring in an Erlenmeyer flask cooled with an ice bath. The reaction medium is subsequently precipitated dropwise, in two installments, from a total volume of 4.5 liters of cooled dichloromethane subjected to vigorous stirring. A white precipitate appears in the medium. The medium is filtered on a sintered glass funnel of No. 4 porosity and then the filter residue is dried under vacuum for 3 days.

Samples are taken at the start and at the end in order to:
determine the kinetics of polymerization (determination of the conversion by moles and by weight by ¹H NMR (d₆-DMSO, 300 MHz);
monitor the change in the number-average molar masses (Mₙ) as a function of the conversion of monomers.

The characteristics of the poly(sodium methacrylate-co-sodium styrenesulfonate) macroinitiator synthesized, after purification, are presented in table 2 below.

TABLE 2

| Time | Conversion | M_{n,}^{a)}ₑₓₚₑᵣᵢₘₑₙₜₐₗ | M_{n,}^{b)}_{theoretical} | | M_{n,}^{c)}ₑₓₚₑᵣᵢₘₑₙₜₐₗ |
|---|---|---|---|---|---|
| (min) | (%) | (g · mol⁻¹) | (g · mol⁻¹) | Iₚ | (g · mol⁻¹) |
| 16 | 10 | 7200 | 1300 | 1.5 | 6350 |

^{a)}Determined by size exclusion chromatography in DMF with 1 g.l⁻¹ of LiBr, with calibration with polymethyl methacrylate, after methylation of the methacrylic acid units to give methyl methacrylate units after purification;
^{b)}Calculated from methyl methacrylate units;
^{c)}Calculated from methacrylic acid units after purification.

The experimental Mₙ is determined by size exclusion chromatography in DMF containing 1 g/l of LiBr, with calibration with polymethyl methacrylate, after methylation of the methacrylic acid units to give methyl methacrylate units. The flow rate is 0.8 ml/min with toluene as flow rate marker. The samples are prepared at a concentration of 5 mg/ml, are filtered on 0.45 μm filters and are analyzed on Polymer Standards Service columns (gram of 30-1000 Å).

The polydispersity index Iₚ is calculated from methyl methacrylate units.

In a second step, 55.7 g of deionized water, 2.29 g of poly(sodium methacrylate-co-sodium styrenesulfonate) macroinitiator (4.54×10⁻³ mol.l_{aq}⁻¹), 23.7 g of 1M sodium hydroxide solution (1 equivalent with respect to the methacrylic acid units) and 0.295 g of Na₂CO₃ (3.5×10⁻² mol.l⁻¹) are introduced into a 250 ml single-necked round-bottomed flask. This mixture is stirred at ambient temperature for approximately 15 minutes until the macroinitiator has completely dissolved, which macroinitiator is then in the poly(sodium methacrylate-co-sodium styrenesulfonate) form. 18.2 g of methyl methacrylate and 1.8 g of styrene are subsequently added (solids content=19.5%) and the mixture is degassed by bubbling with nitrogen at ambient temperature for 30 minutes.

The medium is then introduced into a Parr® reactor, series 5100, equipped with a 300 ml single-shelled glass vessel with an internal diameter of 63 mm and a working height of 102 mm. Stirring is maintained with a magnetically driven stirrer provided with a turbine at 250 rpm. The vessel of the reactor is heated beforehand.

The medium is introduced into the hot reactor under a nitrogen pressure of 3 bar and the time t=0 is launched at 60° C. and is maintained at 90° C. throughout the polymerization. Samples are taken at regular intervals in order:
to determine the kinetics of polymerization by gravimetry (measurement of solids content);
to monitor the change in the number-average molar masses (Mₙ) with the conversion of monomers;
to evaluate the colloidal characteristics of the latex (by TEM).

The characteristics of the latexes withdrawn are presented in table 3 below.

TABLE 3

| Time | Conversion | M_{n, exp}^{a} | Mₙ, theo^{b} | | |
|---|---|---|---|---|---|
| (h) | (%) | (g · mol⁻¹) | (g · mol⁻¹) | Iₚ^{a} | pH |
| 0.25 | 18 | 23 900 | 17 200 | 1.3 | 7.9 |
| 0.5 | 25.5 | 31 600 | 21 350 | 1.24 | — |
| 0.75 | 43.6 | 42 850 | 31 400 | 1.13 | 7.55 |
| 1 | 52 | 46 700 | 36 000 | 1.13 | — |
| 3.1 | 68 | 53 700 | 44 900 | 1.2 | 6.7 |

^{a}Determined by size exclusion chromatography in DMF with 1 g · l⁻¹ of LiBr, with calibration with polymethyl methacrylate, after methylation of the methacrylic acid units to give methyl methacrylate units;
^{b}Calculated from methyl methacrylate units.

The latex obtained at the end of polymerization is white and very viscous.

The appearance of the particles is analyzed by transmission electron microscopy (TEM). This microscope is of JEOL 100 Cx II type at 100 keV equipped with a high resolution CCD camera, Keen View camera from SIS.

### EXAMPLE 3

### Thermal Aging of the Latexes of Crosslinked and Noncrosslinked Filamentous Polymer Particles

Latexes comprising 1% by weight of crosslinked (sample A) and noncrosslinked (sample B) filamentous polymer particles are subjected to thermal aging at 140° C. in an enclosed aqueous medium for several days. The rheological properties and the structure of the particles are monitored over time. The rheological properties of these compositions are measured using a controlled-stress rheometer of Anton Paar MCR 301 type. The flow measurements (viscosity as a function of the shear rate) are carried out at ambient temperature (25° C.) with a Couette or plate-plate geometry (depending on the viscosity range). The thermal aging tests are carried out in a closed reactor, under pressure, in order to keep the water in the liquid state.

The results obtained are presented in the appended FIG. 1 and in table 4.

FIG. 1 represents the variation in the viscosity (Pa·s at 20° C.) as a function of the shear rate (s⁻¹). The symbols used in FIG. 1 have the following meanings:
Δ sample A, 1% in water
□ sample A, 1% in water, after 2 days at 140° C.
◯ sample A, 1% in water, after 4 days at 140° C.
♦ sample B, 1% in water
▴ sample B, 1% in water, after 1 day at 140° C.
x water

TABLE 4

| | | Viscosity of the latexes (1% of fibrillar | | | |
|---|---|---|---|---|---|
| | | micelles) at 25° C. (mPa · s) | | | |
| | | as a function of the shear rate | | | |
| Samples | Thermal aging | 0.1 s⁻¹ | 1 s⁻¹ | 10 s⁻¹ | 100 s⁻¹ |
| Sample B | without | 829 | 171 | 34 | 13 |
| | 1 day at 140° C. | — | — | 2 | 1.8 |
| Sample A | without | 1130 | 95 | 17 | 7 |
| | 2 days at 140° C. | 1420 | 161 | 32 | 9 |
| | 4 days at 140° C. | 1100 | 55 | 19 | 6 |

These results (table 4 and FIG. 1) show that the structural integrity of the crosslinked fiber is retained, even after thermal aging for several days in an aqueous medium. After 4 days spent at 140° C., the latex retains the same rheological behavior as the initial latex (high viscosity at low shear rates and same change in this viscosity as a function of the shear rate). The crosslinking factor is very important since, in the case of a latex consisting of noncrosslinked fibers (obtained according to comparative example 2), the thermal aging degrades the fibrillar structure of the latex and the rheological properties (viscosifying and shear thinning) are lost (the viscosity falls by two orders of magnitude and becomes unchanging as a function of the shear rate).

### Abbreviations

CRP—controlled radical polymerization
P4VP—poly(4-vinylpyridine)
PNaA—poly(sodium acrylate)
SG1—N-tert-butyl-N-[1-diethylphosphono-2,2-dimethylpropyl]
S or Sty—styrene
SS—sodium styrenesulfonate
AA—acrylic acid
PEGA—poly(ethylene glycol) acrylate methyl ether
TEM—transmission electron microscopy
RAFT—reversible addition/fragmentation chain transfer
MAA—methacrylic acid
DMSO—dimethyl sulfoxide
DMF—dimethylformamide
rpm—rotations per minute
f_{0,STY}—initial molar fraction of styrene in the mixture of monomers
f_{0,SS}—initial molar fraction of sodium styrenesulfonate in the mixture of monomers
f_{0,DVP}—initial molar fraction of divinylbenzene in the mixture of monomers
BlocBuilder®-MA-(N-(2-methylpropyl)-N-(1-diethylphosphono-2,2-dimethylpropyl)-O-(2-carboxylprop-2-yl)hydroxylamine

1. A process for increasing resistance to thermal aging of an aqueous or organic solution, said process comprising combining said solution with a latex of filamentous polymer particles, said particles being crosslinked, block copolymers synthesized by controlled radical emulsion polymerization, in the form of cylinders having a length/diameter ratio of greater than 100 and added to said solution at a content by weight of a minimum of 100 ppm.

2. The process as claimed in claim 1, in which said particles are synthesized from at least one hydrophobic monomer and a crosslinking agent in the presence of a living macroinitiator derived from a nitroxide, under the following conditions:
said crosslinked filamentous particles are obtained in an aqueous medium during the synthesis of said block copolymers carried out by heating the reaction medium at a temperature of 60 to 120° C.,
said macroinitiator is water-soluble,
the percentage of the molar mass of the water-soluble macroinitiator in the final block copolymer is between 10 and 50%, and of that:
the degree of conversion of the hydrophobic monomer is at least 50%.

3. The process as claimed in claim 1, in which said particles have a length of greater than 500 nm.

4. The process as claimed in claim 1, in which the hydrophobic monomer is a vinylaromatic monomers, an alkyl, cycloalkyl or aryl acrylate, an alkyl, cycloalkyl, alkenyl or aryl methacrylate, or vinylpyridine.

5. The process as claimed in claim 1, in which the percentage of the molar mass of the water-soluble macroinitiator in the final block copolymer is between 10 and 30%.

6. The process as claimed in claim 1, in which the content by weight of the hydrophilic part making up the final block copolymer is less than 25%.

7. The process as claimed in claim 1, in which said crosslinking comonomer is chosen from divinylbenzenes, trivinylbenzenes, allyl (meth)acrylates, diallyl maleate polyol (meth)acrylates and alkylene glycol di(meth)acrylates which have from 2 to 10 carbon atoms in the carbon-based chain.

8. The process as claimed in claim 1, in in which the crosslinking agent is introduced into the reaction medium at a content of at least 1% by weight and preferably between 5 and 15% by weight, with respect to the weight of hydrophobic monomer.

9. The process as claimed in claim 1, in which said viscosifying composition is obtained by the addition of said filamentous polymer particles to an aqueous or organic solution at a content by weight of 500 to 10 000 ppm.

10. The process as claimed in claim 1, in which said viscosifying composition containing at least 500 ppm of said particles and mixed with water or with brine is injected under pressure into the rock in order to extract hydrocarbons therefrom.

11. The process as claimed in claim 1, in which said viscosifying composition is a thickening composition.

12. The process as claimed in claim 1, in which said viscosifying composition is a composition intended for the preparation of paints.

13. The process as claimed in claim 1, in which said viscosifying composition is a cosmetic composition.
